(19) United States
(12) Patent Application Publication
(10) Pub. No.: US 2026/0229342 A1
(43) Pub. Date: Aug. 6, 2026

(54) AUTOMATIC ANNOTATION OF ENDOSCOPIC VIDEOS

(71) Applicant: GYRUS ACMI, INC D/B/A OLYMPUS SURGICAL TECHNOLOGIES AMERICA, Westborough, MA (US)

(72) Inventor: Dawei Liu, Sharon, MA (US)

(21) Appl. No.: 19/159,073

(22) PCT Filed: Feb. 13, 2024
(86) PCT No.: PCT/US2024/015539

§ 371 Date: Aug. 22, 2025

Related U.S. Application Data
(60) Provisional application No. 63/486,698, filed on Feb. 24, 2023.

(51) Int. Cl. G16H 30/40; A61B 1/05; G06F 40/169; G06F 40/279; G06V 20/40; G10L 15/26; G11B 27/036; G11B 27/34; G16H 10/40

(52) U.S. Cl. CPC G16H 30/40; A61B 1/05; G06F 40/169; G06F 40/279; G06V 20/41; G10L 15/26; G11B 27/036; G11B 27/34; G16H 10/40

(57) ABSTRACT

A method for automatic annotation of individual frames of procedural videos can include receiving, with processing circuitry of a controller, a video stream captured by an endoscopic camera during an endoscopic procedure. The video stream can include a first timestamp. The method can also include, receiving an audio recording captured during the endoscopic procedure. The audio recording can include a second timestamp. The method can also include, receiving a transcribed text from the audio recording. The transcribed text can also the second timestamp. The method can also include, annotating the video stream with the transcribed text by corresponding the transcribed audio and the video stream when the first timestamp and the second timestamp agree.

### PRIORITY CLAIM

This application claims the benefit of priority to U.S. Provisional Patent Application Ser. No. 63/486,698, filed Feb. 24, 2023, the contents of which are incorporated herein by reference in their entirety.

### TECHNICAL FIELD

This disclosure generally relates to endoscopes and, more particularly, to the automatic annotation of individual frames of endoscopy videos.

### BACKGROUND

Conventional endoscopes can be used in a variety of clinical procedures. For example, endoscopes can be used for illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, providing suction passageways for collecting fluids (e.g., saline or other preparations), and the like. Such anatomical regions can include the gastrointestinal tract (e.g., esophagus, stomach, duodenum, pancreaticobiliary duct, intestines, colon, and the like), renal area (e.g., kidney(s), ureter, bladder, urethra), other internal organs (e.g., reproductive systems, sinus cavities, submucosal regions, respiratory tract), and the like.

### BRIEF DESCRIPTION OF THE DRAWINGS

Various examples are illustrated in the figures of the accompanying drawings. Such examples are demonstrative and not intended to be exhaustive or exclusive embodiments of the present subject matter.

FIG. 1 illustrates a schematic diagram of an endoscopy system, according to an example of the present disclosure.

FIG. 2 illustrates a schematic diagram of the imaging and control system of FIG. 1, showing the imaging and control system connected to the endoscope, according to an example of the present disclosure.

FIG. 3 is a block diagram of an example of a control unit for an endoscopic system for automatic annotation of individual frames of endoscopy videos, according to an example of the present disclosure.

FIG. 4 is a flowchart illustrating a method, according to an example of the present disclosure.

FIG. 5 is a flowchart further illustrating the method from FIG. 4, according to an example of the present disclosure.

FIG. 6 is a flowchart further illustrating the method from FIG. 4, according to an example of the present disclosure.

FIG. 7 is a flowchart further illustrating the method from FIG. 4, according to an example of the present disclosure.

FIG. 8 is a flowchart further illustrating the method from FIG. 4, according to an example of the present disclosure.

FIG. 9 is a flowchart further illustrating the method from FIG. 4, according to an example of the present disclosure.

FIG. 10 is a flowchart further illustrating the method from FIG. 4, according to an example of the present disclosure.

FIG. 11 is a flowchart further illustrating the method from FIG. 4, according to an example of the present disclosure.

FIG. 12 is a flowchart further illustrating the method from FIG. 4, according to an example of the present disclosure.

FIG. 13 is a schematic diagram of an example of an annotated image from a video stream captured during a medical procedure.

FIG. 14 is a block diagram illustrating an example of a machine upon which one or more embodiments may be implemented.

### DETAILED DESCRIPTION

Endoscopic videos can be noisy and contain unusable frames caused by camera movement or water spray. For example, colonoscopy videos can contain water bubbles from spray or remaining stool due to insufficient bowel preparation. During a colonoscopy, a polypectomy can be performed when polyps are detected, which can obscure the video stream with medical tools and blood from the polyp removal. Because of the uncertainty in the quality of the video streams in colonoscopy videos, colonoscopy video frames are selected and annotated before they can serve as training data for training algorithms to assist with tasks such as polyp detection or classification.

The selection and annotation these images is a manual process that can be performed post-procedural. For example, training data generation can include endoscopists reviewing hours of recorded videos to manually select a subset of usable frames that correspond to moments when the camera was stable and free of noise, debris, tools, or the like. After manually selecting the subset of usable frames, the endoscopists can annotate the frames with any clinical findings from the videos captured during the colonoscopy. Manually annotating the subset of usable frames can be time and resource consuming, which can also be very expensive. Well annotated image data is critical to the proper training of artificial intelligence systems using machine learning algorithms to assist endoscopists in detecting and classifying anomalies during procedures. The larger the training data set, the better the machine learning algorithms will likely perform after training. Accordingly, the inventors of the present disclosure have discovered a need to enhance efficiency and reduce the costs associated with training data generation for use in medical image analysis.

The present disclosure relates to a endoscopic system that can automatically annotate endoscopic videos. For example, the present disclosure generally relates to a system that can automatically identify usable frames during a medical examination, and annotate the usable frames with information that is extracted from intraprocedural speech uttered by a clinician while the clinician is viewing images during the medical examination. During a medical examination, such as a colonoscopy procedure, the performing clinician tends to speak aloud about the clinical findings or medical procedures performed on the detected abnormalities during the procedure. During a colonoscopy clinicians can find a polyp or other abnormality, and the clinicians tend to mention it aloud (e.g., to their team). In another example, sometimes during a colonoscopy the clinician can perform a polypectomy. Here, the clinician typically talks about the polyp and the removal of such polyp. Lastly, when a colon looks healthy, the clinician typically utters that the colon looks good while performing the colonoscopy. The utterances by the clinician completing the medical procedure are typically to ensure the medical team performing the procedure is informed on the status of the procedure, and know if they should take any intervening steps (e.g., polypectomy, etc.). Therefore, the inventors of the present invention have recognized that the utterances of the clinician performing the medical procedure can contain rich clinical information. The examples of the present disclosure enhance efficiencies with respect to training data generation by extracting this rich information and then using the extracted information to automatically annotate the images that a system determines to be usable frames, thereby creating training data that can be used to train algorithms to perform tasks such as polyp detection and classification.

In an example, the endoscopic system can include a camera connected to a distal portion of an elongated member, a microphone mounted around the endoscope in a position that can capture sounds around the medical procedure, a natural language processor configured to process an audio recording captured by the microphone, and a controller configured to receive signals from the camera, microphone, natural language processor to automatically annotate endoscopic videos.

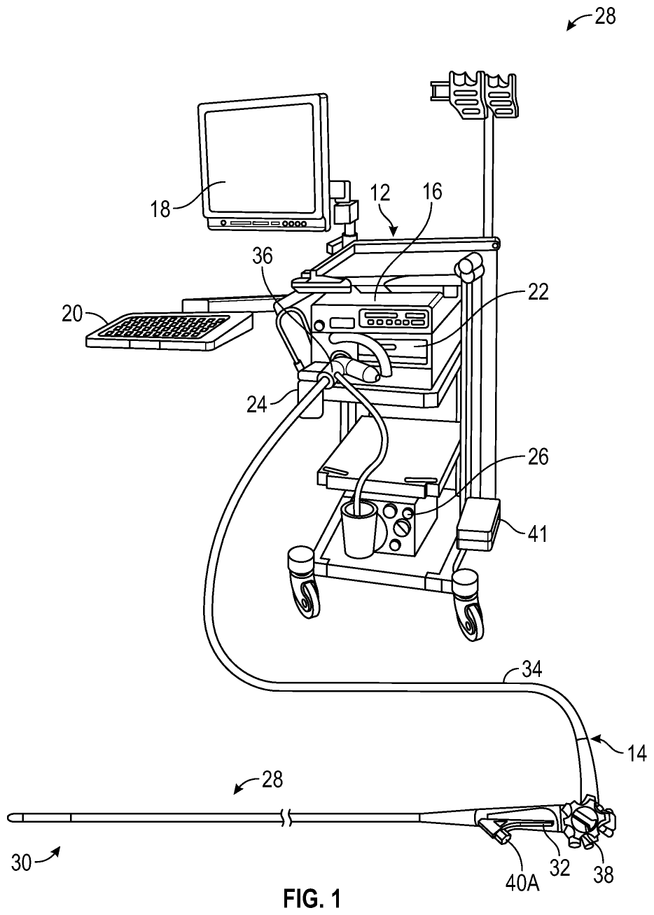

FIG. 1 is a schematic diagram of an endoscopy system 10 that can include an imaging and control system 12 and an endoscope 14. The system 10 is an illustrative example of an endoscopy system suitable for use with the systems, devices, and methods described herein, such as a colonoscope system for automatically annotating endoscopic videos.

The endoscope 14 can be insertable into an anatomical region for imaging or to provide passage of or attachment to (e.g., via tethering) one or more sampling devices for biopsies or therapeutic devices for treatment of a disease state associated with the anatomical region. The endoscope 14 can interface with and connect to imaging and control system 12. The endoscope 14 can also include a colonoscope, though other types of endoscopes can be used with the features and teachings of the present disclosure. The imaging and control system 12 can include a control unit 16, an output unit 18, an input unit 20, a light source unit 22, a fluid source 24, and a suction pump 26.

The imaging and control system 12 can include various ports for coupling with the endoscopy system 10. For example, the control unit 16 can include a data input/output port for receiving data from and communicating data to the endoscope 14. The light source unit 22 can include an output port for transmitting light to the endoscope 14, such as via a fiber optic link. The fluid source 24 can include a port for transmitting fluid to the endoscope 14. The fluid source 24 can include, for example, a pump and a tank of fluid or can be connected to an external tank, vessel, or storage unit. The suction pump 26 can include a port to draw a vacuum from the endoscope 14 to generate suction, such as for withdrawing fluid from the anatomical region into which the endoscope 14 is inserted. The output unit 18 and the input unit 20 can be used by an operator of the endoscopy system 10 to control functions of the endoscopy system 10 and view the output of the endoscope 14. The control unit 16 can also generate signals or other outputs from treating the anatomical region into which the endoscope 14 is inserted. In some examples, the control unit 16 can generate electrical output, acoustic output, fluid output, and the like for treating the anatomical region with, for example, cauterizing, cutting, freezing, and the like.

The endoscope 14 can include an insertion section 28, a functional section 30, and a handle section 32, which can be coupled to a cable section 34 and a coupler section 36. The insertion section 28 can extend distally from the handle section 32, and the cable section 34 can extend proximally from the handle section 32. The insertion section 28 can be elongated and can include a bending section and a distal end to which the functional section 30 can be attached. The bending section can be controllable (e.g., by a control knob 38 on the handle section 32) to maneuver the distal end through tortuous anatomical passageways (e.g., stomach, duodenum, kidney, ureter, etc.). The insertion section 28 can also include one or more working channels (e.g., an internal lumen) that can be elongated and can support the insertion of one or more therapeutic tools of the functional section 30, such as a cholangioscope. The working channel can extend between the handle section 32 and the functional section 30. Additional functionalities, such as fluid passages, guide wires, and pull wires, can also be provided by the insertion section 28 (e.g., via suction or irrigation passageways or the like).

A coupler section 36 can be connected to the control unit 16 to connect to the endoscope 14 to multiple features of the control unit 16, such as the input unit 20, the light source unit 22, the fluid source 24, and the suction pump 26.

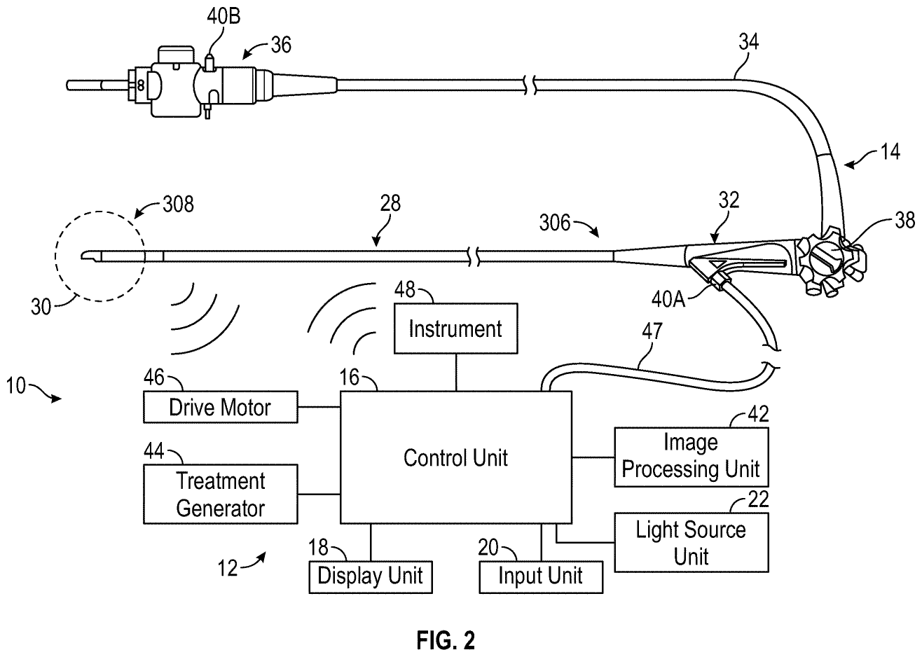

The handle section 32 can include the knob 38 and the port 40A. The knob 38 can be connected to a pull wire or other actuation mechanisms that can extend through the insertion section 28. The port 40A, as well as other ports, such as a port 40B (FIG. 2), can be configured to couple various electrical cables, guide wires, auxiliary scopes, tissue collection devices, fluid tubes, and the like to the handle section 32, such as for coupling with the insertion section 28.

According to examples, the imaging and control system 12 can be provided on a mobile platform (e.g., a cart 41) with shelves for housing the light source unit 22, the suction pump 26, an image processing unit 42 (FIG. 2), etc. Alternatively, several components of the imaging and the control system 12 (shown in FIGS. 1 and 2) can be provided directly on the endoscope 14 to make the endoscope “self-contained.”

The functional section 30 can include components for treating and diagnosing anatomy of a patient. The functional section 30 can include an imaging device, an illumination device, and an elevator. The functional section 30 can further include optically enhanced biological matter and tissue collection and retrieval devices as described herein. For example, the functional section 30 can include one or more electrodes conductively connected to the handle section 32 and functionally connected to the imaging and control system 12 to analyze biological matter in contact with the electrodes based on comparative biological data stored in the imaging and control system 12.

FIG. 2 is a schematic diagram of the endoscopy system 10 of FIG. 1 including the imaging and control system 12 and the endoscope 14. FIG. 2 schematically illustrates components of the imaging and the control system 12 coupled to the endoscope 14, which in the illustrated example includes a colonoscope. The imaging and control system 12 can include the control unit 16, which can include or be coupled to an image processing unit 42, a treatment generator 44, and a drive unit 46, as well as the light source unit 22, the input unit 20, and the output unit 18. The control unit 16 can include, or can be in communication with, an endoscope, a surgical instrument 48, and an endoscopy system, which can include a device configured to engage tissue and collect and store a portion of that tissue and through which imaging equipment (e.g., a camera) can view target tissue via inclusion of optically enhanced materials and components. The control unit 16 can be configured to activate a camera to view target tissue distal of the endoscopy system. Likewise, the control unit 16 can be configured to activate the light source unit 22 to shine light on the surgical instrument 48, which can include select components configured to reflect light in a particular manner, such as enhanced tissue cutters with reflective particles.

The coupler section 36 can be connected to the control unit 16 to connect to the endoscope 14 to multiple features of the control unit 16, such as the image processing unit 42 and the treatment generator 44. In examples, the port 40A can be used to insert another surgical instrument 48 or device, such as a daughter scope or auxiliary scope, into the endoscope 14. Such instruments and devices can be independently connected to the control unit 16 via the cable 47. In examples, the port 40B can be used to connect coupler section 36 to various inputs and outputs, such as video, air, light, and electric.

The image processing unit 42 and light source unit 22 can each interface with the endoscope 14 (e.g., at the functional section 30) by wired or wireless electrical connections. The imaging and control system 12 can accordingly illuminate an anatomical region, collect signals representing the anatomical region, process signals representing the anatomical region, and display images representing the anatomical region on the display unit 18. The imaging and control system 12 can include the light source unit 22 to illuminate the anatomical region using light of desired spectrum (e.g., broadband white light, narrow-band imaging using preferred electromagnetic wavelengths, and the like). The imaging and control system 12 can connect (e.g., via an endoscope connector) to the endoscope 14 for signal transmission (e.g., light output from light source, video signals from imaging system in the distal end, diagnostic and sensor signals from a diagnostic device, and the like).

The fluid source 24 (shown in FIG. 1) can be in communication with control unit 16 and can include one or more sources of air, saline, or other fluids, as well as associated fluid pathways (e.g., air channels, irrigation channels, suction channels, or the like) and connectors (barb fittings, fluid seals, valves, or the like). The fluid source 24 can be utilized as an activation energy for a biasing device or a pressure-applying device of the present disclosure. The imaging and control system 12 can also include the drive unit 46, which can include a motorized drive for advancing a distal section of endoscope 14.

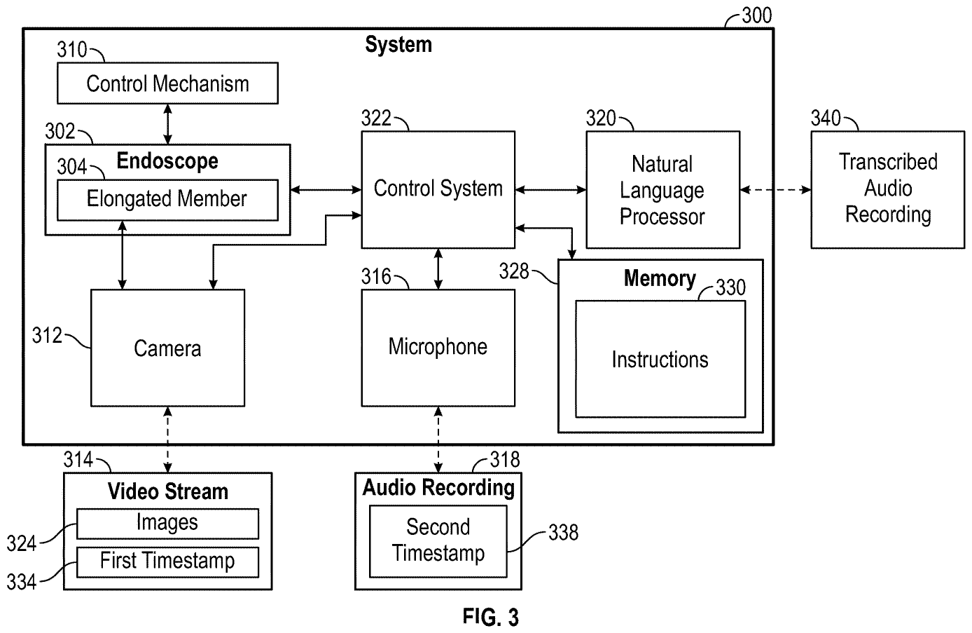

FIG. 3 is a block diagram that describes an example of a system 300 for the automatic annotation of individual frames of colonoscopy videos, according to an example of the present disclosure. The system 300 can include an endoscope 302, a microphone 316, a natural language processor 320, a control system 322, and a memory 328. The endoscope 302 can include an elongated member 304, a control mechanism 310, and a camera 312. As best shown in FIG. 2, the elongated member 304 (e.g., the insertion section 28 and the functional section 30 (FIGS. 1 and 2)) can extend from a proximal portion 306 to a distal portion 308. The elongated member 304 can be insertable into a cavity of a patient.

A control mechanism 310 (e.g., the knob 38 or the handle section 32 (both in FIGS. 1 and 2)) can be coupled to the proximal portion 306 of the elongated member 304. The control mechanism 310 can be configured to navigate the elongated member 304 during the procedure. In examples, the control mechanism 310 can be configured to be manipulated by the doctor or other medical professional completing the medical procedure. In another example, the control mechanism 310 can be controlled by a robot or any other controller that can be used to help navigate the endoscope 302 within a cavity of a patient.

The camera 312 can be attached to the distal portion 308 of the elongated member 304. The camera 312 can be configured to capture a video stream 314 during a medical procedure. The image processing unit 42 (FIG. 2) can process the video stream 314 and display the video stream 314 on the display unit 18 (FIGS. 1 and 2) so doctors, or other medical professionals, can see in front of the distal portion 308 of the elongated member 304 during the medical procedure. The camera 312 can also simultaneously transmit the video stream 314 to multiple components. For example, the camera 312 can transmit the video stream 314 to the display unit to provide a live feed of the video stream 314 on the display for the doctor, the image processing unit or the control system 322 for processing, and the memory 328 for storage of a raw version of the video stream 314. Any example of the video stream 314 can include a first timestamp 334 to help sync the video stream 314 with other signals of the system 300.

One or more of the microphone 316 can be connected to the system 300 to capture an audio recording 318. In an example, the microphone 316 can be mounted on the endoscope 302. For example, the microphone 316 can be mounted on the control mechanism 310. The microphone 316 can be mounted on the handle section 32 (FIG. 1), the knob 38 (also in FIG. 1), or any other location along the endoscope 302 that can detect words spoken by an operator of the endoscope 302 during the medical procedure.

In another example, the microphone 316 can be mounted on a portion of the system 300 detached from the endoscope 302. For example, one or more of the microphone 316 can be mounted on the bed or table that the patient is on during the procedure. One or more of the microphone 316 can be mounted throughout the room, for example, on a wall or any other fixture.

In yet another example, the microphone 316 can be mounted anywhere on the imaging and control system (e.g., the imaging and control system 12 (FIG. 1)) the display or output device (e.g., the output unit 18 (FIG. 1)) the input device (e.g., the input unit 20 (FIG. 1)) or anywhere else on the medical cart (e.g., the cart 41 (FIG. 1)). The system 300 can include one or more of the microphone 316 in wireless communication with the other components of the system 300. For example, the system 300 can include a wireless receiver that is configured to convert sound into an electrical signal that can be transmitted to the natural language processor 320 or the control system 322 for processing.

The audio recording 318 can include spoken words, sounds, or any other noise generated around the system 300 during the procedure. The microphone 316 can transmit the audio recording 318 to the natural language processor 320, the control system 322, or any other component of the system 300 for analysis and compilation. For example, the audio recording 318 can be transmitted by the microphone 316 to more than one component at a time. For example, the microphone 316 can simultaneously transmit the audio recording 318 to the natural language processor 320 or the control system 322 for processing and the memory 328 for storage. Any example of the audio recording 318 can include a second timestamp 338 to help sync the audio recording 318 with other signals around the system 300.

The natural language processor 320 can be configured to receive the audio recording 318 and analyze the audio recording 318 using natural language processing techniques to generate a transcribed audio recording 340. In an example, the natural language processor 320 can run live during the endoscopic procedure. When the natural language processor 320 is running during the endoscopic procedure, the natural language processor 320 can be lagged some degree after the endoscopic procedure so that the natural language processor 320 has data from the audio recording 318 when the natural language processor 320 is initiated. In another example, the natural language processor 320 can be ran offline. For example, the video stream 314 and the audio recording 318 can be sent to the natural language processor 320 after the endoscopic procedure is completed.

In examples, the natural language processor 320 can detect single words from the audio recording 318. In another example, the natural language processor 320 can detect complete sentences, phrases, or paragraphs, which can be grouped together and stored in one or more text files.

The transcribed audio recording 340 can be a complete transcription of the audio recording 318. For example, the transcribed audio recording 340 can include all recognized words found in the audio recording 318 by the natural language processor 320. In another example, the natural language processor 320 or the control system 322 can redact, sort, or otherwise alter the text from the natural language processor 320 to generate a more focused version of the transcribed audio recording 340. The variations of the portions of the audio recording 318 that can be used by the natural language processor 320 to make the transcribed audio recording 340 will be discussed in more detail herein.

The control system 322 (e.g., the control unit 16) can be one or more controllers configured to operate the system 300. The memory 328 can include instructions 330 that when executed by the control system 322, can cause the processing circuitry of the control system 322 to complete operations or procedures. For example, the processing circuitry of the control system 322 can be configured by the instructions 330 to annote one or more images of a video stream by receiving the video stream 314 from the camera 312, receiving the audio recording 318 from the microphone 316, and receiving the transcribed audio recording 340 from the natural language processor 320 and completing procedures as dictated by the instructions 330 to annotate the frames of the endoscopic video. The control system 322 will be discussed in more detail herein.

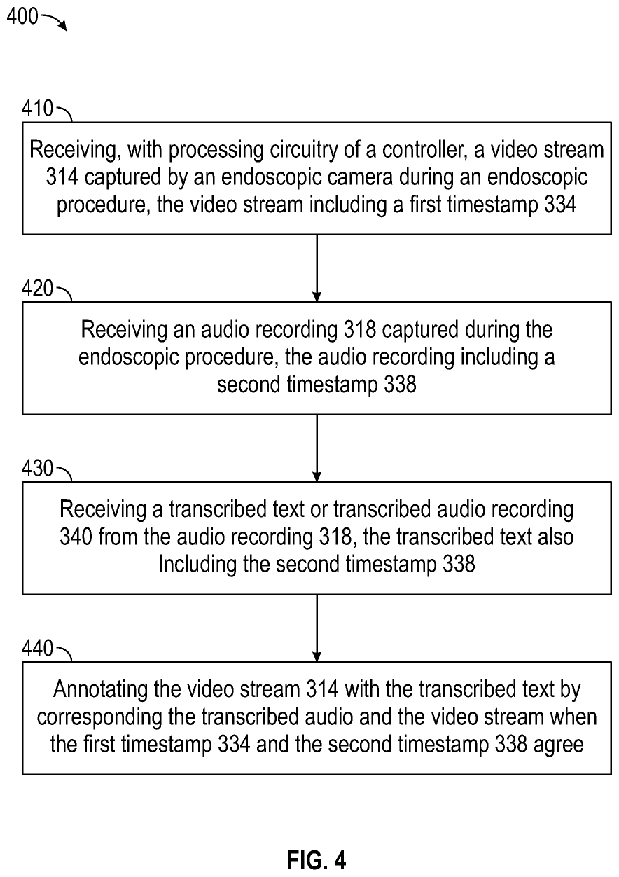

The instructions 330 can then cause the processing circuitry of the control system 322 to complete procedures or tasks. For example, the instructions 330 can guide the control system 322 to annotate one or more images 324 of the video stream 314 with the transcribed text from the transcribed audio recording 340 by corresponding the transcribed audio recording 340 and the video stream 314 when the first timestamp 334 and the second timestamp 338 agree. The first timestamp 334 and the second timestamp 338 can agree when the first timestamp 334 and the second timestamp 338 are the same. In another example, there can be a range, for example, the first timestamp 334 can agree with the second timestamp 338 when the first timestamp 334 and the second timestamp 338 are within a threshold of one another. The one or more annotated images can include a still image of the video stream 314 with annotated text from the transcribed audio recording 340 or the audio recording 318. The instructions 330 and their interactions with the control system 322 will be discussed in more detail herein with reference to FIGS. 4-12.

FIG. 4 is a flowchart that describes a method 400, according to an example of the present disclosure. The method 400 can automatically annotate endoscopic videos. As discussed above with reference to FIG. 3, the system 300 can be used to capture audio recordings, capture video recordings, and generate a transcribed text file from the audio or video recordings while the medical procedure is being performed. In examples, the annotated images can be displayed on a display unit that is visible to the doctor completing the medical procedure, overlayed on the video stream of the medical procedure, transmitted to a database, or stored in memory. The method 400 will be discussed below with reference to FIGS. 4-12.

At step 410, the method 400 can include receiving, with processing circuitry of a controller (e.g., the natural language processor 320 or the control system 322 from FIG. 3), a video stream 314 captured by an endoscopic camera (e.g., the camera 312 from FIG. 3) during an endoscopic procedure. For example, the video stream 314 can be a continuous feed transmitted from a camera on the endoscope. In another example, the video stream 314 can be one or more images that can be spliced together to form the video stream 314. Here, the video stream 314 can be sent to a video processor (e.g., the image processing unit 42 (FIG. 2)) to analyze the video stream 314 and generate one or more images of the video stream 314 that best captures the medical procedure. For example, the one or more images can be clear of debris, blood, tools, or any other obstructions such that the one or more images best show the medical procedure. Each of the one or more images can have the first timestamp 334 such that the time of each of the one or more images can be determined after the procedure.

At step 420, the method 400 can include receiving an audio recording 318 captured during the endoscopic procedure. The audio recording 318 can be one or more signals detected from one or more microphones installed around the procedure room. For example, the audio recording 318 can be a single recording that combines each signal detected from each microphone around the room. In another example, the audio recording 318 can be individual recordings of each recording of the one or more microphones around the procedure room. Regardless, each recording of the audio recording 318 can include the second timestamp 338. The control system 322 can receive the audio recording 318 and transmit the audio recording 318 to one or more components of the system 300, for example, to the natural language processor 320 or to memory 328 for storage.

At step 430, the method 400 can include receiving a transcribed text or transcribed audio recording 340 of the audio recording 318. In examples, the transcribed audio recording 340 can include transcription from any of the audio recording 318. The natural language processor (e.g., natural language processor 320 (FIG. 3), or any other language processor can be connected to the system to transcribe the audio to generate the transcribed audio recording 340. The transcribed audio recording 340 can also include the second timestamp 338.

At step 440, the method 400 can include annotating the video stream 314 with the transcribed audio recording 340 by corresponding the transcribed audio recording 340 and the video stream when the first timestamp 334 and the second timestamp 338 agree. For example, the control system 322 can overlay the video stream or one or more images of the video stream with the transcribed audio recording 340 such that the first timestamp 334 on the transcribed audio recording 340 and the second timestamp 338 of the video stream 314 match.

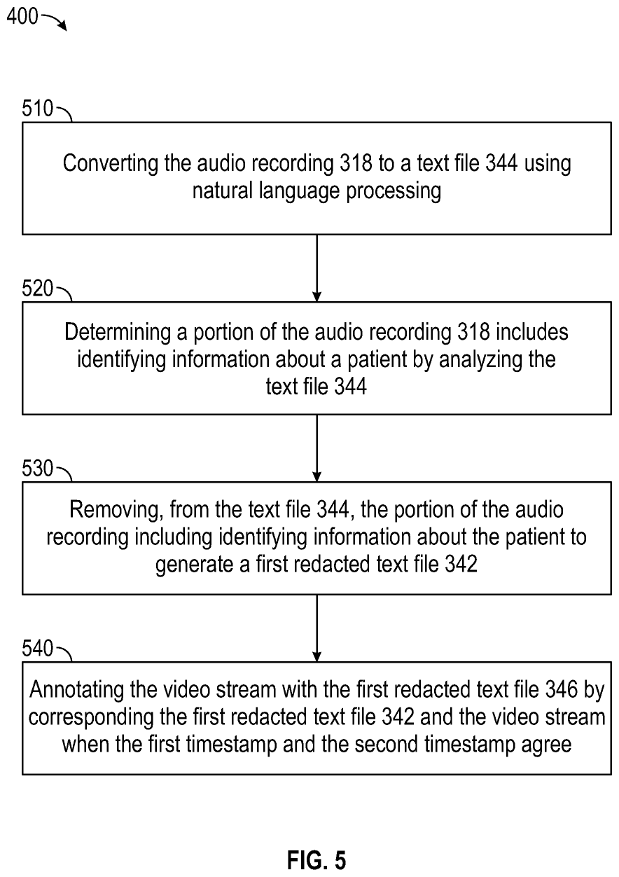

FIG. 5 is a flowchart that further describes the method 400 from FIG. 4, according to an example of the present disclosure. In an example, step 440 of the method 400 from FIG. 4 can optionally include steps 510-540 that can be performed on the processing circuitry of the natural language processor 320 or the control system 322 processing the video stream (e.g., the video stream 314) with a first redacted text file 342.

At step 510, the method 400 can include converting the audio recording to a text file using natural language processing. In an example, the control system 322 can send the audio recording to the natural language processor 320 to generate the first redacted text file 342. The natural language processor 320 can convert the audio recording 318 to a text file 344 (e.g., the transcribed audio recording 340) using natural language processing and can transmit the text file 344 back to the control system 322 or can store the text file 344 in the memory 328 for additional processing.

At step 520, the method 400 can include determining a portion of the audio recording by identifying information about a patient by analyzing the text file. The natural language processor 320 or the control system 322 can analyze the text file 344 to determine a portion of the audio recording 318 includes identifying information about a patient. The identifying information can be any description of the patient that can help identify the patient. For example, the identifying information can include a name, age, race or ethnicity, or any other factor that can be used to identify a patient. In an example, the natural language processor 320 or the control system 322 can be configured to customize words that are redacted from the text file 344. For example, words of profanity, slang, or any other non-professional terms that can affect the training data integrity of the annotated images can be redacted from the text file 344.

At step 530, the method 400 can include removing, from the text file 344, the portion of the audio recording including identifying information about the patient to generate a first redacted text file 342. For example, the natural language processor 320 or the control system 322 can alter the text file 344 by removing the portion of the audio recording 318 that includes identifying information about the patient to generate the first redacted text file 342. As discussed above, the natural language processor 320 or the control system 322 can remove or redact any other words that the natural language processor 320 or the control system 322 is configured to detect and redact. Therefore, the first redacted text file 342 can be a clean text file that is ready to be annotated to generate training data. The control system 322 can save the first redacted text file 342 separately from the text file 344 such that both the text file 344 and the first redacted text file 342 can be processed later. Each of the first redacted text file 342 and the text file 344 can include a timestamp to help synch the text in the first redacted text file 342 and the text file 344 with other samples taken during the medical procedure.

At step 540, the method 400 can include annotating the video stream 314 with the first redacted text file 342 by corresponding the first redacted text file 342 and the video stream 314 when the first time stamp and the second time stamp agree. For example, the control system 322 can then annotate the video stream 314, or one or more images of the video stream 314, with the first redacted text file 342 by corresponding the first redacted text file 346 and the video stream 314 when the first timestamp 334 and the second timestamp 338 agree.

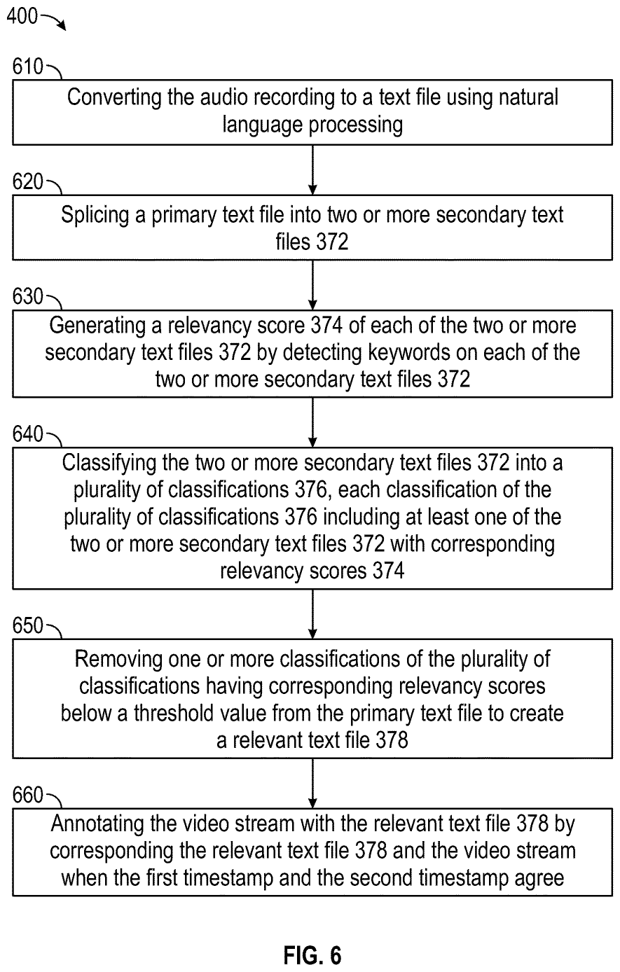

FIG. 6 is a flowchart that further describes additional optional operations performed as part of the method 400 from FIG. 4, according to an example of the present disclosure. In an example, step 440 of the method 400 from FIG. 4 can optionally include the processing circuitry of the controller (e.g., the natural language processor 320 or the control system 322) annotating the video stream (e.g., the video stream 314) with a relevant audio text file by completing steps 610-660.

At step 610, the method 400 can include converting the audio recording to a text file using natural language processing. For example, the method 400 can complete step 510 as discussed with reference to FIG. 5.

At step 620, the method 400 can include splicing a primary text file (e, g, the transcribed audio recording 340 (FIG. 3)) into two or more secondary text files 372. The two or more secondary text files 372 can be spliced from the transcribed audio recording 340 based on timing, context, or any other indicator that can help decipher the utterances of the medical professional during the medical procedure.

At step 630, the method 400 can include generating a relevancy score 374 of each of the two or more secondary text files 372 by detecting keywords on each of the two or more secondary text files 372. The relevancy score 374 can correspond to a relevancy of the each of the two or more secondary text files 372 according to preconfigured keywords. For example, the relevancy score 374 can be configured to increase a relevancy of one of the two or more secondary text files 372 if one or more keywords are present and decrease a relevancy of one of the two or more secondary text files 372 if one or more alternative keywords are present.

At step 640, the method 400 can include classifying the two or more secondary text files 372 into a plurality of classifications 376. Each classification of the plurality of classifications 376 including at least one of the two or more secondary text files 372 with corresponding relevancy scores 374. Here, the two or more secondary text files 372 that have similar relevancy scores 374 can be combined into a classification of the plurality of classifications 376. Such sorting into the classifications can help group or gather the most relevant portions of the two or more secondary text files 372. Alternatively, the control system 322 can group or gather the least relevant portions of the two or more secondary text files 372 and group them into classifications to help eliminate one or more of the two or more secondary text files 372 from being analyzed.

At step 650, the method 400 can include removing one or more classifications of the plurality of classifications 376 having corresponding relevancy scores 374 below a threshold value from the primary text file (e.g., the transcribed audio recording 340), to create a relevant text file 378. For example, a pre-determined threshold value can be selected to filter the most relevant portions of the text file. Removing classifications below this threshold can ensure the quality or relevancy of the remaining classifications.

At step 660, the method 400 can include annotating the video stream (e.g., the video stream 314) with the relevant text file 378 by corresponding the relevant text file 378 and the video stream when the first timestamp and the second timestamp agree. Here, the control system 322 can annotate just the most relevant images. Annotating the most relevant images can decrease the computing time and resources required for the annotation and can decrease an amount of storage required to store the annotated relevant images.

Moreover, annotating the relevant text according to each classification can result in a focused set of annotated figures. For example, a classification can be for a type of polyp or abnormality, a process or technique performed during the procedure, a tool used during the procedure, or the like. Therefore, the focus of the classifications can further help focus the inputs for machine learning to help detect those instances, procedures, or abnormalities using neural networks and artificial intelligence.

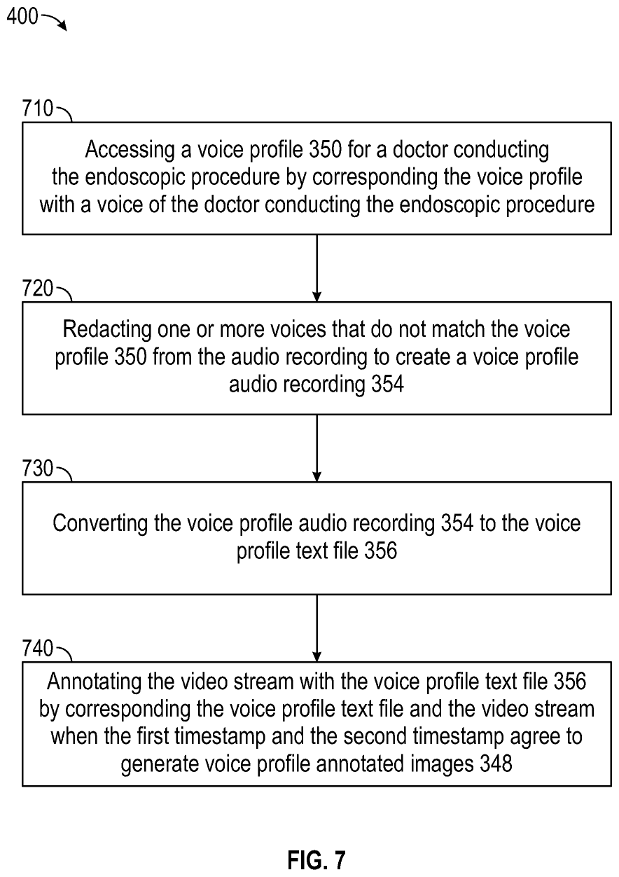

FIG. 7 is a flowchart that further describes additional optional operations that can be performed as part of the method 400 from FIG. 4, according to an example of the present disclosure. In an example, step 440 of the method 400 of FIG. 4 can optionally include the processing circuitry of the controller (e.g., the natural language processor 320 or the control system 322) processing the video stream with a voice profile 350 to generate voice profile annotated images 348 by including steps 710 to 740.

In an example, step 710 of the method 400 can include accessing a voice profile 350 for a doctor conducting the endoscopic procedure by corresponding the voice profile with the voice of the doctor conducting the endoscopic procedure. The voice profile 350 can be stored on the memory 328 or any other memory of the system 300 and can be compared to the voices found on each audio recording to find the correct voice profile for the medical professional completing the medical procedure. The natural language processor 320 or the control system 322 can access the voice profile 350 for a doctor conducting the endoscopic procedure by corresponding the voice profile 350 with a voice of the doctor conducting the endoscopic procedure.

At step 720, the method 400 can include redacting one or more voices that do not match the voice profile 350 from the audio recording, to create a voice profile audio recording 354. For examples, The natural language processor 320 or the control system 322 can redact one or more voices that do not match the voice profile 350 from the audio recording 318 or the transcribed audio recording 340 to create a voice profile audio recording 354.

The voice profile audio recording 354 can include the voices of people that match one or more of the voice profile 350. In an example, the voice profile 350 can be maintained only for medical professionals with proper credentials (e.g., licensed doctors, nurse practitioners, physician assistants, or the like) to ensure that captured words are of a qualified person. In another example, each person that works around the system 300 can have a unique version of the voice profile 350, and the voice profile 350 can be tagged with restrictions or clearances as appropriate to match the credentials of the respective person from which the voice profile 350 was generated. Therefore, the voice profile audio recording 354 can include tags, indicia, or other labels corresponding to the medical licensing or credentials of the voice profile 350 contained therein.

In examples, the voice profile audio recording 354 can be stored in the memory with the audio record, the raw transcribed audio recording, and the video stream. The voice profile audio recording 354 can also include a timestamp that can help the control system 322 sync the 354//with the video stream 314 or one or more images of the video stream 314.

At step 730, the method 400 can include converting the voice profile audio recording 354 to a voice profile text file 356. For example, the natural language processor 320 or the control system 322 can convert the voice profile audio recording 354 to a voice profile text file 356 using natural language processing techniques. Similar to the voice profile audio recording 354, the control system 322 can know the one or more of the voice profile 350 contained on the voice profile text file 356, which can include tags, indicia, or labels corresponding to the medical licensing or credentials of the voice profile 350 contained therein. The voice profile text file 356 can be stored with the voice profile audio recording 354, the video stream, or any other files from the system 300. The voice profile text file 356 can also include the timestamp to help the control system 322 sync the voice profile text file 356 with other files from the system 300.

At step 740, the method 400 can include annotating the video stream with the voice profile text file 356 by corresponding the voice profile text file 356 and the video stream 314, or one or more images, when the first timestamp and the second timestamp agree. In an example, the natural language processor 320 or the control system 322 can annotate the one or more images 324 from the video stream 314 with the voice profile text file 356 to generate the voice profile annotated images 348 by corresponding the voice profile text file 356 and the video stream 314 when the first timestamp 334 and the second timestamp 338 agree. The voice profile annotated images 348 can contain the indicia, labels, or other indications of the credentials or clearances of the respective voice profile 350 contained therein, and can be stored alone, or with other data of the system 300, on the memory 328 for future reference. The filtered nature of isolating the voice profile 350 of a grouping of medical professionals, or an individual doctor, can provide information rich images that can help focus the review of the one or more images, or help focus the inputs for machine learning.

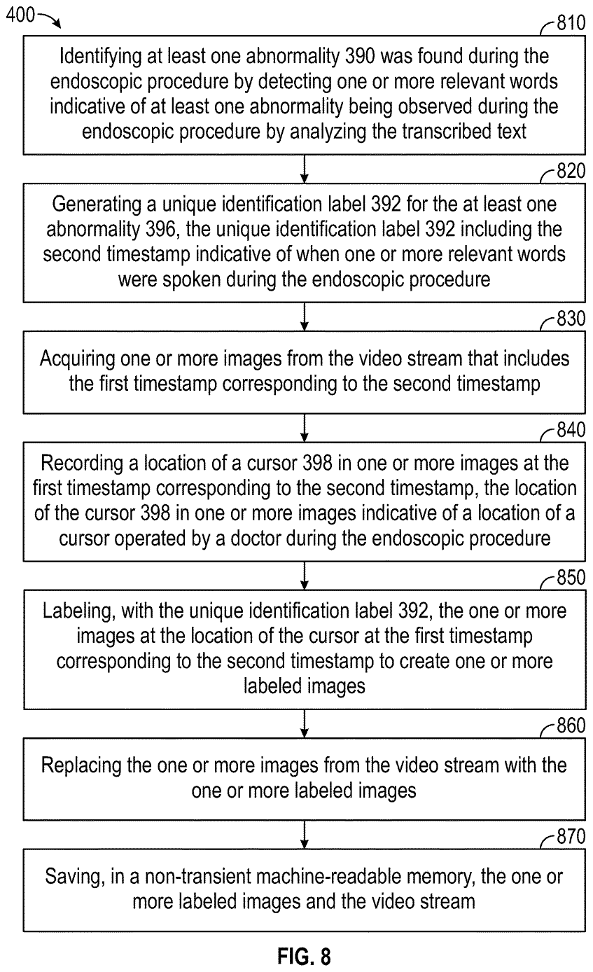

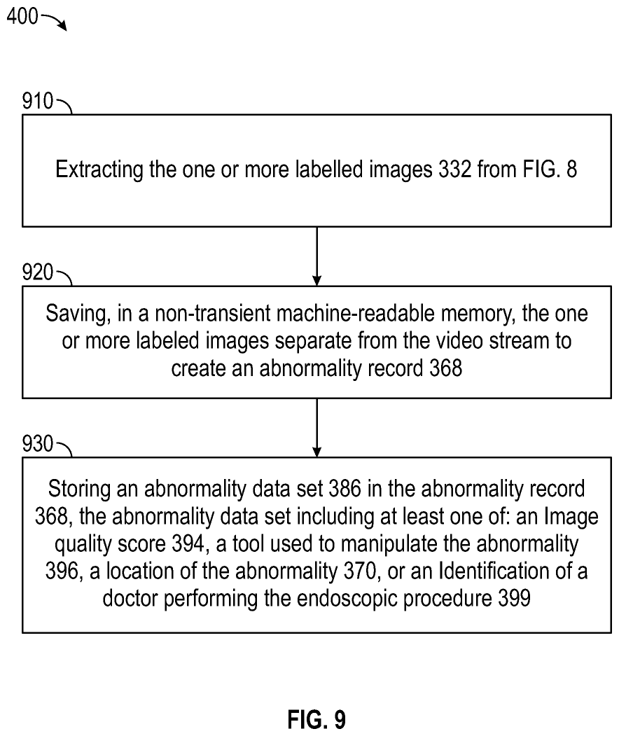

FIG. 8 is a flowchart that further describes additional optional operations that can be performed as part of the method 400 from FIG. 4, according to an example of the present disclosure. In an example, the method 400 can optionally include steps 810-870 to generate one or more labeled images 332.

At step 810, the method 400 can include identifying at least one abnormality was found during the endoscopic procedure by detecting one or more relevant words indicative of at least one abnormality being observed during the endoscopic procedure by analyzing the transcribed text. For example, the control system 322 can run instructions 330 to generate one or more labeled images 332. For example, the control system 322 can annotate the video stream 314 by identifying at least one abnormality 390 was found during the endoscopic procedure by detecting one or more relevant words, or keywords (e.g., the one or more keywords 360) indicative of at least one abnormality 390 being observed during the endoscopic procedure by analyzing the transcribed text (e.g., the transcribed audio recording 340).

At step 820, the method 400 can include generating a unique identification label 392 for the at least one abnormality 390. The unique identification label 392 can include the second timestamp (e.g., the second timestamp 338) indicative of when one or more relevant words were spoken during the endoscopic procedure. The unique identification label 392 can identify the type of polyp, and can be used to reference that particular polyp in future scans or medical procedures. The unique identification label 392 can also be used to track tests or pathology results of a polyp after it has been removed. In another example, the unique identification label 392 can be used to track changes in size, shape, color, texture, or any other physical feature detected during a medical procedure of the identified polyp.

At step 830, the method 400 can include acquiring one or more images 324 from the video stream 314 that include the first timestamp 334 that can correspond to the second timestamp 338. In such an example, the second timestamp 338 can be indicative of when one or more relevant words were spoken during the endoscopic procedure. Thus, the identified polyp can likely be found on the one or more images at, or around, that corresponding timestamp.

At step 840, the method 400 can include instructions 330 configured the control system 322 to record a location of the cursor 398 during the medical procedure. The location of the cursor 398 can be the location of the cursor that the operator (e.g., the doctor, nurse, or the like) of the system 300 is using to perform the medical procedure. For example, the location of the cursor 398 can include a timestamp (e.g., the first timestamp 334 or the second timestamp 338). The location of the cursor 398 can be saved on the memory 328 and later recalled for processing or overlaying.

At step 850, the method 400 can include labeling, with the unique identification label 392, the one or more images 324 with the location of the cursor 398 at the first timestamp 334 corresponding to the second timestamp 338, to create one or more labeled images 332. For example, the location of the cursor 398 can be annotated, overlayed, projected thereon, or the like, onto one or more images 324 by corresponding the location of the cursor 398 at the first timestamp 334 with the one or more images 324 at the second timestamp 338 to create one or more labeled images 332. The one or more labeled images 332 can include the unique identification label 392 and the location of the cursor 398 to help direct the review of the reviewing doctor, or help focus the machine learning during the machine learning process.

At step 860, the method 400 can include replacing the one or more images from the video stream 314 with the one or more labeled images 332. In an example, the one or more labeled images 332 can then replace the one or more images 324 from the video stream 314 with the one or more labeled images 332. Here, the video stream 314 inclusive of the one or more images 324 can be projected onto a display in the operating room. In another example, the original stream of the video stream 314 can be shown on a first display, and the video stream 314 inclusive of the one or more images 324 can be shown on another display within the operating room.

At step 870, the method 400 can include saving, in a non-transient machine-readable memory, the one or more labeled images 332 and the video stream. The video stream 314 inclusive of the one or more images 324 can be saved separately from the video stream 314 to preserve the video stream 314 and the video stream 314 with the one or more images 324.

FIG. 9 is a flowchart that further describes additional optional operations that can be performed as part of the method 400 from FIG. 4, according to an example of the present disclosure. In an example, the method 400 can optionally include steps 910-930.

At step 910, the method 400 can include extracting the one or more labeled images 332. For example, the control system 322 can extract one or more of the one or more labeled images 332 from the steps of the method 400 discussed in FIG. 8. Thus, the one or more labeled images 332 can be separated from the one or more images of the video stream 314 or any non-labeled images.

At step 920, the method 400 can include saving, in a non-transient computer-readable memory, the one or more labeled images separate from the video stream to create an abnormality record 368. For example, the control system 322 can save the one or more labeled images 332 (FIG. 8) separate from the video stream 314 in the memory 328 to create an abnormality record 368. The abnormality record 368 can include a record for each of the abnormalities that have the unique identification label 392. For example, if multiple of the one or more labeled images 332 contain an image of a single polyp, each of those one or more labeled images 332 with the corresponding unique identification label 392 can be saved together in the abnormality record 368. Therefore, each of the unique identification label 392 can include a unique abnormality record 368 that can be used to track changes, tests, or other results, of the abnormality. Moreover, the abnormality record 368 that correspond to a corresponding grouping or subset of the unique identification label 392 can be used for further machine learning on the specific type or grouping of the types of polyps captured in the respective one or more labeled images 332.

At step 930, the method 400 can include storing an abnormality data set 386 in the abnormality record 368. The abnormality data set 386 including at least one of: an image quality score 394, a tool used to manipulate abnormality 396, a location of the abnormality, or an identification of a doctor performing the endoscopic procedure 399. The abnormality data sets 386 can be used to determine best practices, or potentially suggest best practices to the doctor of future procedures that comes across one or more abnormalities of similar qualities.

The image quality score 394 can be configured to provide a confidence level, or a image quality score that can be used to filter out obstructed or blurry images. For example, a higher image quality score can be indicative of a clear image with little obstruction. A lower image quality score can be indicative of blurriness, obstruction, a lack of focus or clarity of the image. In examples, the control system 322 or any other image processor, can be configured to run an algorithm that can analyze and determine the image quality score 394.

The tool used to manipulate abnormality 396 can be a type of scalpel, blade, suction, suture, stitch, or any other instrument that can engaged with one or more of the abnormalities within a body. For example, the tool used to manipulate the abnormality 396 can be captured to help suggest tools to the doctors performing future medical procedures as they come across a corresponding abnormality.

The identification of a doctor performing the endoscopic procedure 399 can be used to ask questions of the medical service provider. Moreover, the identification of a doctor performing endoscopic procedure 399 can be used to learn the preferences of the doctor such that the system 300 can learn the tools, procedures, or steps that that the respective doctor prefers when they encounter different abnormalities. This understanding by the system 300 can help the system 300 recommend procedures, tools, or steps that operating doctor prefers for future medical procedures. The identification of a doctor performing the endoscopic procedure 399 can also help direct the review of the abnormalities after the medical examination is complete.

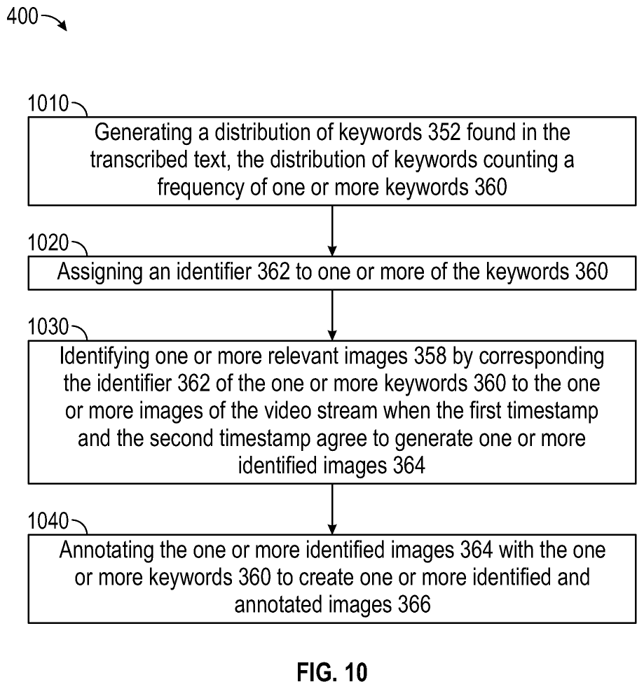

FIG. 10 is a flowchart that further describes additional optional operations that can be performed as part of the method 400 from FIG. 4, according to an example of the present disclosure. In an example, steps 810-830 from FIG. 8 of the method 400, can optionally include steps 1010-1040 to enable the natural language processor 320 or the control system 322 to identify relevant images 358.

At step 1010, the method 400 can include generating a distribution of keywords 352 found in the transcribed text, For example, the instructions 330 can configure the processing circuitry of the natural language processor 320 or the control system 322 to generate a distribution of keywords 352. Each keyword of the distribution of keywords 352 can found in the transcribed text (e.g., the transcribed audio recording 340).

In examples, the natural language processor 320 or the control system 322 can generate the distribution of keywords 352 by counting a frequency of one or more keywords 360. Other natural language processing techniques to sort the one or more keywords 360 can be used to generate the distribution of the keywords 352. For example, a relevancy score, a confidence score, or any other analysis can be completed by the natural language processor 320 or the control system 322 to find the relevancy of the one or more keywords 360. The one or more keywords 360 can include words that can indicate an abnormality found during the procedure. For example, the one or more keywords 360 can include, “polyp,” “abnormality,” “look here,” “right there,” any other word that can signal an abnormality is encountered during the procedure, or the like.

At step 1020, the method 400 can include assigning an identifier 362 to one or more of the keywords 360. The natural language processor 320 or the control system 322 can also assign an identifier 362 to one or more of the keywords 360. The identifier 362 can be indicative of types or styles of the one or more keywords 360 found in the audio recording or text file. For example, if a polyp was detected, the identifier 362 can indicate a polyp or other abnormality was found.

At step 1030, the method 400 can include the instructions configuring the processing circuitry of the natural language processor 320 or the control system 322 to identify one or more relevant images 358 by corresponding the identifier 362 of one or more of the keywords 360 to the one or more images 324 of the video stream 314 when the first timestamp 334 and the second timestamp 338 agree to generate one or more identified images 364. By matching the first timestamp 334 and the second timestamp 338 the natural language processor 320 or the control system 322 can find one or more images that can contain a visual depiction of the abnormality detected from the utterances of the doctor.

At step 1040, the method 400 can include annotating the one or more identified images 364 with the one or more of keywords 360 to create one or more identified and annotated images. In an example, the natural language processor 320 or the control system 322 can annotate the one or more identified images 364 with the identifier 362 to one or more of the keywords 360 to create one or more identified and annotated images 366. The one or more identified and annotated images 366 can be displayed on a display within the room, which can help with further analysis of the abnormality during the medical procedure. In another example, the one or more identified and annotated images 366 can be saved on a memory (e.g., the memory 328) or in a file directory for later recall or analysis.

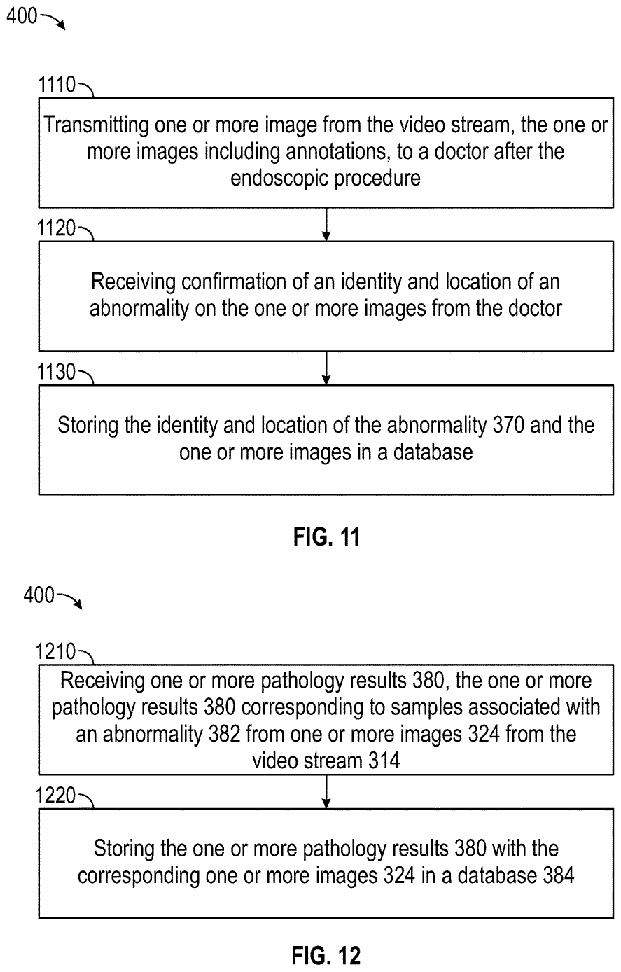

FIG. 11 is a flowchart that further describes additional optional operations that can be performed as part of the method 400 from FIG. 4, according to an example of the present disclosure. In an example, the method 400 of FIG. 4 can optionally include steps 1110-1130.

At step 1110, the method 400 can include transmitting one or more images from the video stream, the one or more images including annotations, to a doctor after the endoscopic procedure. To confirm the identity and the location of the abnormality 370, the control system 322 can transmit the one or more identified and annotated images 366 to a doctor. For example, the control system 322 can transmit the one or more identified and annotated images 366 to a doctor via e-mail, charting software, or any other physical or electronic means that allow the doctor to analyze the identity and location of the abnormality 370 in the one or more identified and annotated images 366. Such review can be completed within the operating room, or later at any computer that can communicate with the system 300.

At step 1120, the method 400 can include receiving confirmation of an identity and location of an abnormality on the one or more images from the doctor. The control system 322 can receive confirmation of an identity and location of an abnormality 370 on the one or more images 324 from the doctor.

At step 1130, the method 400 can include storing the identity and location of the abnormality and the one or more images in a database. Once the control system 322 receives confirmation of the identity and location of the abnormality 370 the control system 322 can send the image to a file directory used for training or machine learning. In another example, the control system 322 can save the confirmation to the abnormality data set, abnormality record, or to a patients medical records.

FIG. 12 is a flowchart that further describes additional optional operations that can be performed as part of the method 400 from FIG. 4, according to an example of the present disclosure. In an example, the method 400 from FIG. 4 can optionally include steps of 1210-1220.

At step 1210, the method 400 can include receiving one or more pathology results 380, the one or more pathology results 380 corresponding to samples associated with an abnormality 382 from one or more images 324 from the video stream 314. The pathology results can provide information as to whether the abnormality is diseased, or further diagnosis of the abnormality 382.

At step 1220, the method 400 can include storing the one or more pathology results with the corresponding one or more images in a database. In an example, the control system 322 can receive one or more pathology results 380. The one or more pathology results 380 can correspond to samples associated with an abnormality 382 from one or more images 324 from the video stream 314. The control system 322 can then store the one or more pathology results 380 with the corresponding one or more images 324 in a database 384.

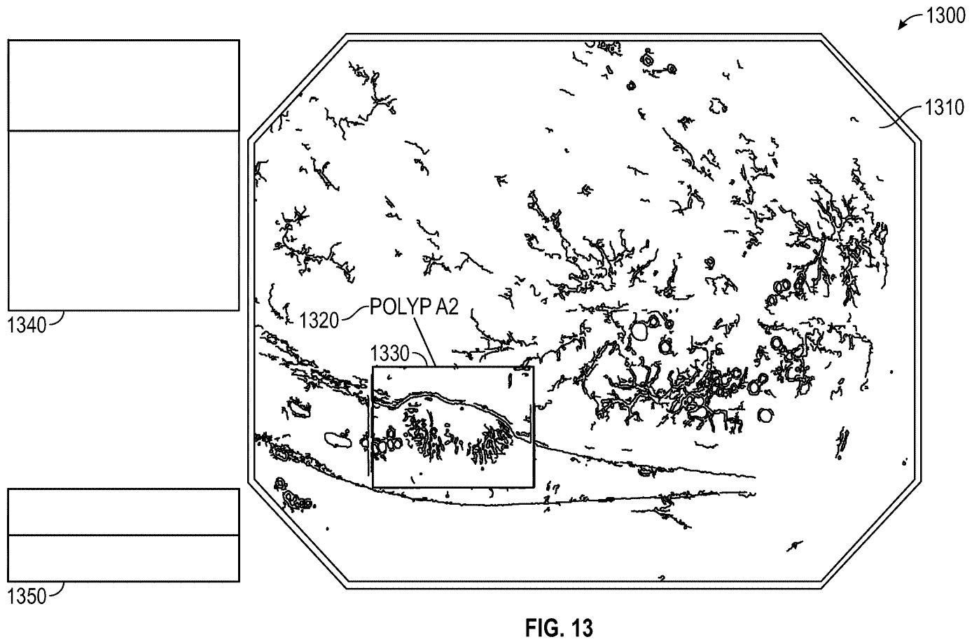

FIG. 13 illustrates a schematic diagram of an example of an annotated image 1300. The annotated image 1300 can for example be any of the annotated images discussed herein, and can include an image 1310, an annotation 1320, a marking box 1330, a polyp identification box 1340, and a process identification box 1350.

The image 1310 can be an individual frame from the video stream captured by the camera during the endoscopic procedure. The image 1310 can be from a timestamp that corresponds a timestamp of a spoken keyword, or any other indicator of an abnormality found during the procedure. The controller can analyze the image 1310 to ensure the most clear version of the video stream is used from a timestamp that corresponds to the found abnormality. For example, the image 1310 can be an image of the video stream from before or after the corresponding timestamp where the abnormality was found if that image can provide a more clear image or better view of the found abnormality.

The annotation 1320 can be located on the image 1310, as is shown in FIG. 13. In another example, the annotation 1320 can be off to the side of the 1310, for example, in the polyp identification box 1340, the process identification box 1350, or any area around the image 1310. The annotation 1320 can be of a spoken keyword, or a unique identifier generated for the abnormality. The annotation 1320 can help identify a location of the abnormality encountered during the medical procedure.

The marking box 1330 can be overlayed the 1310 to help identify the abnormality found. For example, the marking box 1330 can help a doctor that is reviewing the annotated image quickly find the abnormality to improve the review of the abnormality. In another example, the marking box 1330 can help the machine learning algorithm focus on the abnormality to improve the quality of learning.

The polyp identification box 1340 can include information about the abnormality from the medical procedure or from review by the doctor after the medical procedure. For example, the polyp identification box 1340 can include annotation of utterances made by the medical professional before and after the timestamp of the keyword being spoken. In another example, the polyp identification box 1340 can include notes typed in by the doctor after the doctor reviews the annotated image 1300. The information provided in the polyp identification box 1340 can help improve the machine learning by providing additional information about the annotated image 1300, which can help sort the annotated image 1300 into groupings of similar findings to improve the information being provided for the machine learning.

The process identification box 1350 can include process information about the medical procedure. For example, the process identification box 1350 can include a timestamp of the video stream that the image is captured from, a timestamp that the keyword was recognized, a confidence level or the identification of the poly, and any other processing information of the medical procedure that can be beneficial to know after the procedure is completed. The process identification box 1350 can also include manufacturing information or model numbers for the equipment used to perform the medical procedure.

The example of annotated image 1300 shown in FIG. 13 is just one example of the annotated image 1300. This example, including the information presented thereon is in no way intended to limit the scope of the invention. Rather, the provided information is intended to be a single example of the annotated image 1300 that the systems described herein can generate.

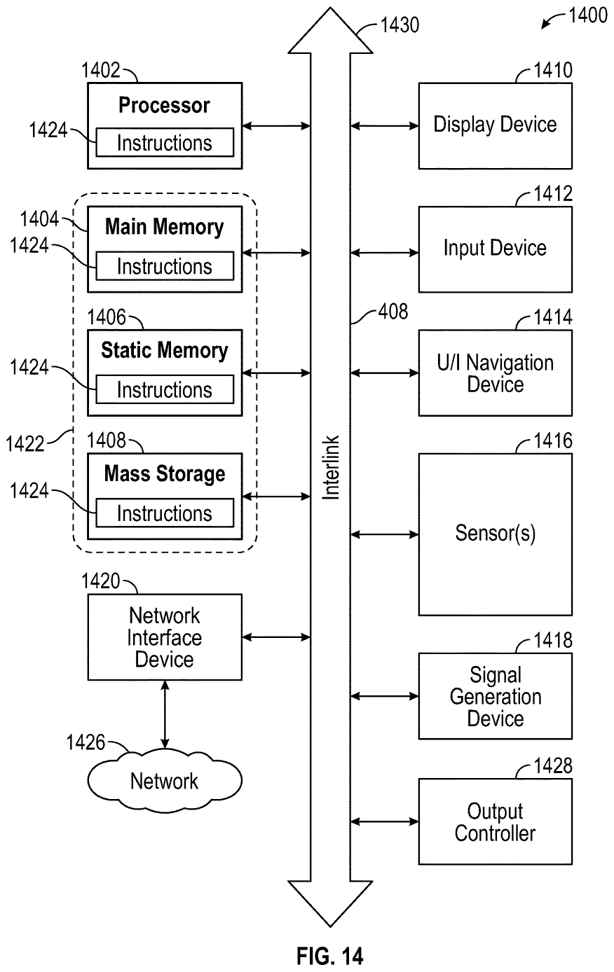

FIG. 14 illustrates a block diagram of an example machine 1400 upon which any one or more of the techniques (e.g., methodologies) discussed herein may perform. Examples, as described herein, may include, or may operate by, logic or a number of components, or mechanisms in the machine 1400. Circuitry (e.g., processing circuitry) is a collection of circuits implemented in tangible entities of the machine 1400 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1400 follow.

In alternative embodiments, the machine 1400 may operate as a standalone device or may be connected (e.g., networked) to other machines. In a networked deployment, the machine 1400 may operate in the capacity of a server machine, a client machine, or both in server-client network environments. In an example, the machine 1400 may act as a peer machine in peer-to-peer (P2P) (or other distributed) network environment. The machine 1400 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (Saas), other computer cluster configurations.

The machine (e.g., computer system) 1400 may include a hardware processor 1402 (e.g., a central processing unit (CPU), a graphics processing unit (GPU), a hardware processor core, or any combination thereof), a main memory 1404, a static memory (e.g., memory or storage for firmware, microcode, a basic-input-output (BIOS), unified extensible firmware interface (UEFI), etc.) 1406, and mass storage 1408 (e.g., hard drives, tape drives, flash storage, or other block devices) some or all of which may communicate with each other via an interlink (e.g., bus) 1430. The machine 1400 may further include a display unit 1410, an alphanumeric input device 1412 (e.g., a keyboard), and a user interface (UI) navigation device 1414 (e.g., a mouse). In an example, the display unit 1410, input device 1412 and UI navigation device 1414 may be a touch screen display. The machine 1400 may additionally include a storage device (e.g., drive unit) 1408, a signal generation device 1418 (e.g., a speaker), a network interface device 1420, and one or more sensors 1416, such as a global positioning system (GPS) sensor, compass, accelerometer, or other sensor. The machine 1400 may include an output controller 1428, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1402, the main memory 1404, the static memory 1406, or the mass storage 1408 may be, or include, a machine readable medium 1422 on which is stored one or more sets of data structures or instructions 1424 (e.g., software) embodying or utilized by any one or more of the techniques or functions described herein. The instructions 1424 may also reside, completely or at least partially, within any of registers of the processor 1402, the main memory 1404, the static memory 1406, or the mass storage 1408 during execution thereof by the machine 1400. In an example, one or any combination of the hardware processor 1402, the main memory 1404, the static memory 1406, or the mass storage 1408 may constitute the machine readable media 1422. While the machine readable medium 1422 is illustrated as a single medium, the term “machine readable medium” may include a single medium or multiple media (e.g., a centralized or distributed database, and/or associated caches and servers) configured to store the one or more instructions 1424.

The term “machine readable medium” may include any medium that is capable of storing, encoding, or carrying instructions for execution by the machine 1400 and that cause the machine 1400 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 1422 may be representative of the instructions 1424, such as instructions 1424 themselves or a format from which the instructions 1424 may be derived. This format from which the instructions 1424 may be derived may include source code, encoded instructions (e.g., in compressed or encrypted form), packaged instructions (e.g., split into multiple packages), or the like. The information representative of the instructions 1424 in the machine readable medium 1422 may be processed by processing circuitry into the instructions to implement any of the operations discussed herein. For example, deriving the instructions 1424 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 1424.

In an example, the derivation of the instructions 1424 may include assembly, compilation, or interpretation of the information (e.g., by the processing circuitry) to create the instructions 1424 from some intermediate or preprocessed format provided by the machine readable medium 1422. The information, when provided in multiple parts, may be combined, unpacked, and modified to create the instructions 1424. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 1424 may be further transmitted or received over a communications network 1426 using a transmission medium via the network interface device 1420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 702.11 family of standards known as Wi-Fi®, IEEE 702.15.4 family of standards, peer-to-peer (P2P) networks, among others. In an example, the network interface device 1420 may include one or more physical jacks (e.g., Ethernet, coaxial, or phone jacks) or one or more antennas to connect to the communications network 1426. In an example, the network interface device 1420 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1400, and includes digital or analog communications signals or other intangible medium to facilitate communication of such software. A transmission medium is a machine readable medium.

The following, non-limiting examples, detail certain aspects of the present subject matter to solve the challenges and provide the benefits discussed herein, among others.

Example 1 is a method for automatic annotation of individual frames of procedural videos, the method comprising: receiving, with processing circuitry of a controller, a video stream captured by an endoscopic camera during an endoscopic procedure, the video stream including a first timestamp; receiving an audio recording captured during the endoscopic procedure, the audio recording including a second timestamp; receiving a transcribed text from the audio recording, the transcribed text including the second timestamp; and annotating the video stream with the transcribed text by corresponding the transcribed audio and the video stream when the first timestamp and the second timestamp agree.

In Example 2, the subject matter of Example 1 includes, converting the audio recording to a text file using natural language processing.

In Example 3, the subject matter of Example 2 includes, wherein annotating includes the processing circuitry of the controller processing the video stream with a first redacted text file by: converting the audio recording to a text file using natural language processing; determining a portion of the audio recording includes identifying information about a patient by analyzing the text file; removing, from the text file, the portion of the audio recording including identifying information about the patient to generate the first redacted text file; and annotating the video stream with the first redacted text file by corresponding the first redacted text file and the video stream when the first timestamp and the second timestamp agree.

In Example 4, the subject matter of Examples 1-3 includes, wherein annotating includes the processing circuitry of the controller processing the video stream with a voice profile text file by: accessing a voice profile for a doctor conducting the endoscopic procedure by corresponding the voice profile with a voice of the doctor conducting the endoscopic procedure; redacting one or more voices that do not match the voice profile from the audio recording to create a voice profile audio recording; converting the voice profile audio recording to the voice profile text file; and annotating the video stream with the voice profile text file by corresponding the voice profile text file and the video stream when the first timestamp and the second timestamp agree.

In Example 5, the subject matter of Examples 2-4 includes, wherein annotating includes the processing circuitry of the controller processing the video stream with a relevant audio text file by: splicing a primary text file into two or more secondary text files; and generating a relevancy score of each of the two or more secondary text files by detecting keywords on each of the two or more secondary text files.

In Example 6, the subject matter of Example 5 includes, wherein annotating includes the processing circuitry of the controller processing the video stream with a relevant audio text file by: classifying the two or more secondary text files into a plurality of classifications, each classification of the plurality of classifications including at least one of the two or more secondary text files with corresponding relevancy scores; removing one or more classifications of the plurality of classifications having corresponding relevancy scores below a threshold value from the primary text file to create a relevant text file; and annotating the video stream with the relevant text file by corresponding the relevant text file and the video stream when the first timestamp and the second timestamp agree.

In Example 7, the subject matter of Examples 1-6 includes, wherein the processing circuitry of the controller generates one or more labeled images by: identifying at least one abnormality was found during the endoscopic procedure by detecting one or more relevant words indicative of at least one abnormality being observed during the endoscopic procedure by analyzing the transcribed text; generating a unique identification label for the at least one abnormality, the unique identification label including the second timestamp indicative of when one or more relevant words were spoken during the endoscopic procedure; and acquiring one or more images from the video stream that includes the first timestamp corresponding to the second timestamp.

In Example 8, the subject matter of Example 7 includes, recording a location of a cursor in one or more images at the first timestamp corresponding to the second timestamp, the location of the cursor in one or more images indicative of a location of a pointer operated by a doctor during the endoscopic procedure.

In Example 9, the subject matter of Example 8 includes, labeling, with the unique identification label, the one or more images at the location of the cursor at the first timestamp corresponding to the second timestamp, to create one or more labeled images.

In Example 10, the subject matter of Example 9 includes, replacing the one or more images from the video stream with the one or more labeled images; and saving, in a non-transient machine-readable memory, the one or more labeled images and the video stream.

In Example 11, the subject matter of Examples 9-10 includes, extracting the one or more labeled images; saving, in a non-transient machine-readable memory, the one or more labeled images separate from the video stream to create an abnormality record; and storing an abnormality data set in the abnormality record, the abnormality data set including at least one of: an image quality score, a tool used to manipulate abnormality, a location of the abnormality, or an identification of a doctor performing the endoscopic procedure.

In Example 12, the subject matter of Examples 7-11 includes, generating a distribution of keywords found in the transcribed text, the distribution of keywords counting a frequency of one or more keywords; assigning an identifier to one or more of the keywords; identifying one or more images by corresponding the identifier of one or more of the keywords to the one or more images of the video stream when the first timestamp and the second timestamp agree; and annotating the identified one or more images with the identifier to one or more of the keywords to create one or more identified and annotated images.

In Example 13, the subject matter of Examples 1-12 includes, transmitting one or more images from the video stream, the one or more images including annotations, to a doctor after the endoscopic procedure; receiving confirmation of an identity and location of an abnormality on the one or more images from the doctor; and storing the identity and location of the abnormality and the one or more images in a database.

In Example 14, the subject matter of Examples 1-13 includes, receiving one or more pathology results, the one or more pathology results corresponding to samples associated with an abnormality from one or more images from the video stream; and storing the one or more pathology results with the corresponding one or more images in a database.

Example 15 is a system for automatic annotation of individual frames of procedural videos, the system comprising: an endoscope comprising: an elongated member including a distal portion, the elongated member comprising: a camera attached to the distal portion, the camera capturing a video stream during a procedure, the video stream including a first timestamp; a microphone configured to capture an audio recording of sounds around the system during the procedure, the audio recording including a second timestamp; a natural language processor configured to receive the audio recording and a transcribed audio recording, the transcribed audio recording including the second timestamp; a memory including instructions; and a controller including processing circuitry that, when in operation, is configured by the instructions to: receive the video stream from the camera; receive the audio recording from the microphone; receive a transcribed text from the natural language processor; and annotate the video stream with the transcribed text by corresponding the transcribed audio and the video stream when the first timestamp and the second timestamp agree.

In Example 16, the subject matter of Example 15 includes, wherein to annotate the video stream, the processing circuitry of the controller processes the video stream with a first redacted text file by: converting the audio recording to a text file using natural language processing; determining a portion of the audio recording includes identifying information about a patient by analyzing the text file; removing, from the text file, the portion of the audio recording including identifying information about the patient to generate the first redacted text file; and annotating the video stream with the first redacted text file by corresponding the first redacted text file and the video stream when the first timestamp and the second timestamp agree.

In Example 17, the subject matter of Examples 15-16 includes, wherein to annotate the video stream, the processing circuitry of the controller processing the video stream with a voice profile text file by: accessing a voice profile for a doctor conducting the endoscopic procedure by corresponding the voice profile with a voice of the doctor conducting the endoscopic procedure; redacting one or more voices that do not match the voice profile from the audio recording to create a voice profile audio recording; converting the voice profile audio recording to the voice profile text file; and annotating the video stream with the voice profile text file by corresponding the voice profile text file and the video stream when the first timestamp and the second timestamp agree.

In Example 18, the subject matter of Examples 15-17 includes, wherein to annotate the video stream, the processing circuitry of the controller generates one or more labeled images by: identifying at least one abnormality was found during the endoscopic procedure by detecting one or more relevant words indicative of at least one abnormality being observed during the endoscopic procedure by analyzing the transcribed text; generating a unique identification label for the at least one abnormality, the unique identification label including the second timestamp indicative of when one or more relevant words were spoken during the endoscopic procedure; and acquiring one or more images from the video stream that includes the first timestamp corresponding to the second timestamp.

In Example 19, the subject matter of Example 18 includes, wherein the processing circuitry of the controller is configured by the instructions to: record a location of a cursor in one or more images at the first timestamp corresponding to the second timestamp, the location of the cursor in one or more images indicative of a location of a pointer operated by a doctor during the endoscopic procedure; label, with the unique identification label, the one or more images at the location of the cursor at the first timestamp corresponding to the second timestamp, to create one or more labeled images.

In Example 20, the subject matter of Examples 15-19 includes, wherein the processing circuitry of the controller is configured by the instructions to: generate a distribution of keywords found in the transcribed text, the distribution of keywords counting a frequency of one or more keywords; assign an identifier to one or more of the keywords; identify one or more images by corresponding the identifier of one or more of the keywords to the one or more images of the video stream when the first timestamp and the second timestamp agree; and annotate the identified one or more images with the identifier to one or more of the keywords to create one or more identified and annotated images.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. A method for automatic annotation of individual frames of procedural videos, the method comprising:
receiving, with processing circuitry of a controller, a video stream captured by an endoscopic camera during an endoscopic procedure, the video stream including a first timestamp;
receiving an audio recording captured during the endoscopic procedure, the audio recording including a second timestamp;
receiving a transcribed text from the audio recording, the transcribed text including the second timestamp; and
annotating the video stream with the transcribed text by corresponding the transcribed text and the video stream when the first timestamp and the second timestamp agree.

2. The method of claim 1, comprising:
converting the audio recording to a text file using natural language processing.

3. The method of claim 2, wherein annotating includes the processing circuitry of the controller processing the video stream with a first redacted text file by:
converting the audio recording to a text file using natural language processing;
determining a portion of the audio recording includes identifying information about a patient by analyzing the text file;
removing, from the text file, the portion of the audio recording including identifying information about the patient to generate the first redacted text file; and
annotating the video stream with the first redacted text file by corresponding the first redacted text file and the video stream when the first timestamp and the second timestamp agree.

4. The method of claim 1, wherein annotating includes the processing circuitry of the controller processing the video stream with a voice profile text file by:
accessing a voice profile for a doctor conducting the endoscopic procedure by corresponding the voice profile with a voice of the doctor conducting the endoscopic procedure;
redacting one or more voices that do not match the voice profile from the audio recording to create a voice profile audio recording;
converting the voice profile audio recording to the voice profile text file; and
annotating the video stream with the voice profile text file by corresponding the voice profile text file and the video stream when the first timestamp and the second timestamp agree.

5. The method of claim 2, wherein annotating includes the processing circuitry of the controller processing the video stream with a relevant audio text file by:
splicing a primary text file into two or more secondary text files; and
generating a relevancy score of each of the two or more secondary text files by detecting keywords on each of the two or more secondary text files.

6. The method of claim 5, wherein annotating includes the processing circuitry of the controller processing the video stream with a relevant audio text file by:
classifying the two or more secondary text files into a plurality of classifications, each classification of the plurality of classifications including at least one of the two or more secondary text files with corresponding relevancy scores;
removing one or more classifications of the plurality of classifications having corresponding relevancy scores below a threshold value from the primary text file to create a relevant text file; and
annotating the video stream with the relevant text file by corresponding the relevant text file and the video stream when the first timestamp and the second timestamp agree.

7. The method of claim 1, wherein the processing circuitry of the controller generates one or more labeled images by:
identifying at least one abnormality was found during the endoscopic procedure by detecting one or more relevant words indicative of at least one abnormality being observed during the endoscopic procedure by analyzing the transcribed text;
generating a unique identification label for the at least one abnormality, the unique identification label including the second timestamp indicative of when one or more relevant words were spoken during the endoscopic procedure; and
acquiring one or more images from the video stream that includes the first timestamp corresponding to the second timestamp.

8. The method of claim 7, comprising:
recording a location of a cursor in one or more images at the first timestamp corresponding to the second timestamp, the location of the cursor in one or more images indicative of a location of a pointer operated by a doctor during the endoscopic procedure.

9. The method of claim 8, comprising:
labeling, with the unique identification label, the one or more images at the location of the cursor at the first timestamp corresponding to the second timestamp, to create one or more labeled images.

10. The method of claim 9, comprising:
replacing the one or more images from the video stream with the one or more labeled images; and
saving, in a non-transient machine-readable memory, the one or more labeled images and the video stream.

11. The method of claim 9, comprising:
extracting the one or more labeled images;
saving, in a non-transient machine-readable memory, the one or more labeled images separate from the video stream to create an abnormality record; and
storing an abnormality data set in the abnormality record, the abnormality data set including at least one of: an image quality score, a tool used to manipulate abnormality, a location of the abnormality, or an identification of a doctor performing the endoscopic procedure.

12. The method of claim 7, comprising:
generating a distribution of keywords found in the transcribed text, the distribution of keywords counting a frequency of one or more keywords;
assigning an identifier to one or more of the keywords;
identifying one or more images by corresponding the identifier of one or more of the keywords to the one or more images of the video stream when the first timestamp and the second timestamp agree; and
annotating the identified one or more images with the identifier to one or more of the keywords to create one or more identified and annotated images.

13. The method of claim 1, comprising:
transmitting one or more images from the video stream, the one or more images including annotations, to a doctor after the endoscopic procedure;
receiving confirmation of an identity and location of an abnormality on the one or more images from the doctor; and
storing the identity and location of the abnormality and the one or more images in a database.

14. The method of claim 1, comprising:
receiving one or more pathology results, the one or more pathology results corresponding to samples associated with an abnormality from one or more images from the video stream; and
storing the one or more pathology results with the corresponding one or more images in a database.

15. A system for automatic annotation of individual frames of procedural videos, the system comprising:
an endoscope comprising:
an elongated member including a distal portion, the elongated member comprising:
a camera attached to the distal portion, the camera capturing a video stream during a procedure, the video stream including a first timestamp;
a microphone configured to capture an audio recording of sounds around the system during the procedure, the audio recording including a second timestamp;
a natural language processor configured to receive the audio recording and a transcribed audio recording, the transcribed audio recording including the second timestamp;
a memory including instructions; and
a controller including processing circuitry that, when in operation, is configured by the instructions to:
receive the video stream from the camera;
receive the audio recording from the microphone;
receive a transcribed text from the natural language processor; and
annotate the video stream with the transcribed text by corresponding the transcribed audio and the video stream when the first timestamp and the second timestamp agree.

16. The system of claim 15, wherein to annotate the video stream, the processing circuitry of the controller processes the video stream with a first redacted text file by:
converting the audio recording to a text file using natural language processing;
determining a portion of the audio recording includes identifying information about a patient by analyzing the text file;
removing, from the text file, the portion of the audio recording including identifying information about the patient to generate the first redacted text file; and
annotating the video stream with the first redacted text file by corresponding the first redacted text file and the video stream when the first timestamp and the second timestamp agree.

17. The system of claim 15, wherein to annotate the video stream, the processing circuitry of the controller processing the video stream with a voice profile text file by:
accessing a voice profile for a doctor conducting the endoscopic procedure by corresponding the voice profile with a voice of the doctor conducting the endoscopic procedure;
redacting one or more voices that do not match the voice profile from the audio recording to create a voice profile audio recording;
converting the voice profile audio recording to the voice profile text file; and
annotating the video stream with the voice profile text file by corresponding the voice profile text file and the video stream when the first timestamp and the second timestamp agree.

18. The system of claim 15, wherein to annotate the video stream, the processing circuitry of the controller generates one or more labeled images by:
identifying at least one abnormality was found during the endoscopic procedure by detecting one or more relevant words indicative of at least one abnormality being observed during the endoscopic procedure by analyzing the transcribed text;
generating a unique identification label for the at least one abnormality, the unique identification label including the second timestamp indicative of when one or more relevant words were spoken during the endoscopic procedure; and
acquiring one or more images from the video stream that includes the first timestamp corresponding to the second timestamp.

19. The system of claim 18, wherein the processing circuitry of the controller is configured by the instructions to:
record a location of a cursor in one or more images at the first timestamp corresponding to the second timestamp, the location of the cursor in one or more images indicative of a location of a pointer operated by a doctor during the endoscopic procedure;
label, with the unique identification label, the one or more images at the location of the cursor at the first timestamp corresponding to the second timestamp, to create one or more labeled images;
replace the one or more images from the video stream with the one or more labeled images;
extract the one or more labeled images;
save, in the memory, the one or more labeled images separate from the video stream to create an abnormality record; and
store an abnormality data set in the abnormality record, the abnormality data set including at least one of: an image quality score, a tool used to manipulate abnormality, a location of the abnormality, or an identification of a doctor performing the endoscopic procedure.

20. The system of claim 15, wherein the processing circuitry of the controller is configured by the instructions to:
generate a distribution of keywords found in the transcribed text, the distribution of keywords counting a frequency of one or more keywords;
assign an identifier to one or more of the keywords;
identify one or more images by corresponding the identifier of one or more of the keywords to the one or more images of the video stream when the first timestamp and the second timestamp agree;
annotate the identified one or more images with the identifier to one or more of the keywords to create one or more identified and annotated images;
transmit one or more images from the video stream, the one or more images including annotations, to a doctor after the endoscopic procedure;
receive confirmation of an identity and location of an abnormality on the one or more images from the doctor;
receive one or more pathology results, the one or more pathology results corresponding to samples associated with an abnormality from one or more images from the video stream; and
store the identity and location of the abnormality, the one or more images, and the pathology results with the corresponding one or more images in a database.
